(19) United States
(12) Patent Application Publication
(10) Pub. No.: US 2006/0018970 A1
(43) Pub. Date: Jan. 26, 2006

(54) Enoximone formulations and their use in the treatment of cardiac hypertrophy and heart failure

(75) Inventors: Michael Bristow, Cherry Hills Village, CO (US); Richard Gorczynski, Westminster, CO (US); Norve Webb, Loveland, OH (US); Jack Domet, Cincinnati, OH (US); Dhiren Shah, Leawood, KS (US); Paul Jarosz, Westminster, CO (US)

Correspondence Address: FULBRIGHT & JAWORSKI L.L.P., 600 CONGRESS AVE., SUITE 2400, AUSTIN, TX, 78701, US

(73) Assignees: Myogen, Inc.; Aventis Pharmaceuticals Inc.

(21) Appl. No.: 11/010,830

(22) Filed: Dec. 13, 2004

Related U.S. Application Data
(60) Provisional application No. 60/529,374, filed on Dec. 12, 2003.

(51) Int. Cl. A61K 31/4166; A61K 9/14

(52) U.S. Cl. 424/489; 514/389

(57) ABSTRACT

The present invention provides formulations of the drug enoximone that include 40% to 80% non-ionic surfactant. Also provided a methods of treating heart failure and cardiac hypetrophy with these formulations.

This application claims benefit of priority to U.S. Provisional Application Ser. No. 60/529,374, filed Dec. 12, 2003, the entire contents of which are hereby incorporated by reference.

### BACKGROUND OF THE INVENTION

1. Field of the Invention

The present invention relates generally to the fields of cardiology and medicine. More particularly, it concerns formulations of the drug enoximone for use in treating cardiac hypertrophy and heart failure.

2. Description of Related Art

Cardiac hypertrophy is an adaptive response of the heart to many forms of cardiac disease, including hypertension, mechanical load abnormalities, myocardial infarction, valvular dysfunction, certain cardiac arrhythmias, endocrine disorders and genetic mutations in cardiac contractile protein genes. While the hypertrophic response is thought to be an initially compensatory mechanism that augments cardiac performance, sustained hypertrophy is maladaptive and frequently leads to ventricular dilation and the clinical syndrome of heart failure. Accordingly, cardiac hypertrophy has been established as an independent risk factor for cardiac morbidity and mortality (Levy et al., 1990).

Treatment with pharmacological agents still represents the primary mechanism for reducing or eliminating the manifestations of heart failure. Diuretics constitute the first line of treatment for mild-to-moderate heart failure. Unfortunately, many of the commonly used diuretics (e.g., the thiazides) have numerous adverse effects. For example, certain diuretics may increase serum cholesterol and triglycerides. Moreover, diuretics are generally ineffective for patients suffering from severe heart failure.

If diuretics are ineffective, vasodilatory agents may be used; the angiotensin converting (ACE) inhibitors (e.g., enalopril and lisinopril) not only provide symptomatic relief, they also have been reported to decrease mortality (Young et al., 1989). Again, however, the ACE inhibitors are associated with adverse effects that result in their being contraindicated in patients with certain disease states (e.g., renal artery stenosis). Similarly, inotropic agent therapy (i.e., a drug that improves cardiac output by increasing the force of myocardial muscle contraction) is associated with a panoply of adverse reactions, including gastrointestinal problems and central nervous system dysfunction.

Thus, the currently used pharmacological agents have severe shortcomings in particular patient populations. The availability of new, safe and effective agents would undoubtedly benefit patients who either cannot use the pharmacological modalities presently available, or who do not receive adequate relief from those modalities.

### SUMMARY OF THE INVENTION

Thus, in accordance with the present invention, there is provided a pharmaceutical formulation comprising enoximone and about 40-80% non-ionic surfactant by weight, wherein the primary particle size of said enoximone is less than 10 μm on average. The non-ionic surfactant may comprise greater than about 40% of the formulation, greater than about 45% of the formulation, greater than about 50% of the formulation, greater than about 55% of the formulation, greater than about 60% of the formulation, greater than about 64% of the formulation, greater than about 65% of the formulation, greater than about 66% of the formulation, greater than about 67% of the formulation, greater than about 70% of the formulation, greater than about 75% of the formulation, or about 80% of the formulation. Specifically, the non-ionic surfactant comprises about 66% of the formulation. The non-ionic surfactant may comprise about 45-80 % by weight, about 45-75% by weight, about 45-70% by weight, about 45-66% by weight, about 50-80% by weight, about 50-75% by weight, about 50-70% by weight, about 50-66% by weight, about 55-80% by weight, about 55-75% by weight, about 55-70% by weight, about 55-66% by weight, about 60-80% by weight, about 60-75% by weight, about 60-70% by weight, about 60-66% by weight.

The non-ionic surfactant may comprise of one or more of glyceryl monooleates, polyoxethylene sorbitan fatty acid esters, sorbitan esters, polyvinyl alcohols, ethoxylated sorbitan esters, ethoxylated fatty acids, poloxamers, polyglycolized glycerides, olyoxyethylene alkyl ethers, polyoxyethylene castor oil derivatives, or Vitamin E tocopheryl polyethylene glycol succinate. The non-ionic surfactant may be Polysorbate-80. The enoximone particles may be micronized, but in all cases will be present in a particle size of less than about 10 microns, as particle size impacts bioavailability. Particle size may also be uniformly less than about 9 microns, less than about 8 microns, less than about 7 microns, less than about 6 microns, less than about 5 microns, less than about 4 microns, less than about 3 microns, less than about 2 microns, or less than about 1 micron. The formulation may yield an in vitro dissolution profile in simulated gastric fluid solution of not more than 25% of enoximone content is 15 min, and not less that 45% of enoximone content in 30 min, and not less than 70% of enoximone content in 45 min.

Also provided are dosage formulations of enoximone comprising about 40-80% non-ionic surfactant and about 20-60 milligrams of enoximone, wherein the primary particle size of said enoximone is less than 10 μm on average. The non-ionic surfactant may comprise greater than about 40% of the formulation, greater than about 45% of the formulation, greater than about 50% of the formulation, greater than about 55% of the formulation, greater than about 60% of the formulation, greater than about 64% of the formulation, greater than about 65% of the formulation, greater than about 66% of the formulation, greater than about 67% of the formulation, greater than about 70% of the formulation, greater than about 75% of the formulation, or about 80% of the formulation. The non-ionic surfactant may comprise about 66% of the formulation. The dosage formulation may comprise about 20 milligrams, about 25 milligrams, about 30 milligrams, about 35 milligrams, about 40 milligrams, about 45 milligrams, about 50 milligrams, about 55 milligrams, or about 60 milligrams of enoximone. The non-ionic surfactant may comprise of one or more of glyceryl monooleates, polyoxethylene sorbitan fatty acid esters, sorbitan esters, polyvinyl alcohols, ethoxylated sorbitan esters, ethoxylated fatty acids, poloxamers, polyglycolized glycerides, olyoxyethylene alkyl ethers, polyoxyethylene castor oil derivatives, or Vitamin E tocopheryl polyethylene glycol succinate. The non-ionic surfactant may be Polysorbate-80. The enoximone may be micronized, for example, into uniform particle sizes of no larger than 10 microns, or the enoximone particles may be less than about 9 microns, less than about 8 microns, less than about 7 microns, less than about 6 microns, less than about 5 microns, less than about 4 microns, less than about 3 microns, less than about 2 microns, or less than about 1 micron. The enoximone dosage may consists of 25 mg or 50 mg of enoximone.

Also provided are pharmaceutical formulations that, upon chronic or steady state dosing, yield a variety of pharmacokinetic parameters. As used herein, “dosing” shall refer to dosing after normalization of drug levels has been achieved. Provided are:
a pharmaceutical formulation comprising enoximone and 66% Polysorbate-80, wherein the primary particle size of said enoximone is less than 10 μm on average, and wherein said formulation yields a blood level of enoximone of about 1 to about 150 ng/ml immediately after dosing, about 20 to about 400 ng/ml at about 2 hours post-dosing, about 15 to about 200 ng/ml at about 4 hours post-dosing, about 1 to about 150 ng/ml at 8 hours post-dosing; in other embodiments the formulation may yield a blood level of enoximone of 5 to about 160 ng/ml immediately after dosing, about 20 to about 400 ng/ml at about 2 hours post-dosing, about 15 to about 200 ng/ml at about 4 hours post-dosing, about 1 to about 150 ng/ml at 8 hours post-dosing; in another embodiment the formulation may yield a blood level of enoximone of 5 to about 160 ng/ml immediately after dosing, about 30 to about 500 ng/ml at about 2 hours post-dosing, about 20 to about 300 ng/ml at about 4 hours post-dosing, about 5 to about 200 ng/ml at 6 hours post-dosing; in another embodiment the formulation may yield a blood level of enoximone of 5 to about 150 ng/ml immediately after dosing, about 30 to about 400 ng/ml at about 2 hours post-dosing, about 20 to about 200 ng/ml at about 4 hours post-dosing, about 5 to about 150 ng/ml at 8 hours post-dosing; in another embodiment the formulation may yield a blood level of enoximone of 7 to about 149 ng/ml immediately after dosing, about 60 to about 385 ng/ml at about 2 hours post-dosing, about 40 to about 270 ng/ml at about 4 hours post-dosing, about 5 to about 150 ng/ml at 8 hours post-dosing; in another embodiment the formulation may yield a blood level of enoximone of 5 to about 150 ng/ml immediately after dosing, about 50 to about 390 ng/ml at about 2 hours post-dosing, about 40 to about 270 ng/ml at about 4 hours post-dosing, about 15 to about 170 ng/ml at 6 hours post-dosing; in another embodiment the formulation may yield a blood level of enoximone of 7 to about 150 ng/ml immediately after dosing, about 60 to about 385 ng/ml at about 2 hours post-dosing, about 40 to about 270 ng/ml at about 4 hours post-dosing, about 20 to about 170 ng/ml at 6 hours post-dosing; in another embodiment the formulation may yield a blood level of enoximone of 5 to about 150 ng/ml immediately after dosing, about 50 to about 390 ng/ml at about 2 hours post-dosing, about 40 to about 270 ng/ml at about 4 hours post-dosing, about 15 to about 170 ng/ml at 8 hours post-dosing; in another embodiment the formulation may yield a blood level of enoximone of 7 to about 150 ng/ml immediately after dosing, about 60 to about 385 ng/ml at about 2 hours post-dosing, about 40 to about 270 ng/ml at about 4 hours post-dosing, about 20 to about 170 ng/ml at 8 hours post-dosing;
a pharmaceutical formulation comprising enoximone and 66% Polysorbate-80, wherein the primary particle size of said enoximone is less than 10 μm on average, and wherein said formulation yields a blood level of enoximone sulfoxide of about 40 to about 800 ng/ml immediately after dosing, about 80 to about 1500 ng/ml at about 2 hours post-dosing, about 80 to about 1500 ng/ml at about 4 hours post-dosing, about 40 to about 800 ng/ml at about 8 hours post-dosing; in another embodiment the formulation may yield a blood level of enoximone sulfoxide of 50 to about 900 ng/ml immediately after dosing, about 200 to about 1700 ng/ml at about 2 hours post-dosing, about 150 to about 1500 ng/ml at about 4 hours post-dosing, about 50 to about 900 ng/ml at 8 hours post-dosing; in another embodiment the formulation may yield a blood level of enoximone sulfoxide of 50 to about 900 ng/ml immediately after dosing, about 200 to about 1700 ng/ml at about 2 hours post-dosing, about 150 to about 1500 ng/ml at about 4 hours post-dosing, about 50 to about 900 ng/ml at 6 hours post-dosing; in another embodiment the formulation may yield a blood level of enoximone sulfoxide of 50 to about 800 ng/ml immediately after dosing, about 200 to about 1500 ng/ml at about 2 hours post-dosing, about 150 to about 1500 ng/ml at about 4 hours post-dosing, about 50 to about 800 ng/ml at 86 hours post-dosing; in another embodiment the formulation may yield a blood level of enoximone sulfoxide of 60 to about 800 ng/ml immediately after dosing, about 300 to about 1500 ng/ml at about 2 hours post-dosing, about 200 to about 1350 ng/ml at about 4 hours post-dosing, about 50 to about 800 ng/ml at 8 hours post-dosing; in another embodiment the formulation may yield a blood level of enoximone sulfoxide of 50 to about 800 ng/ml immediately after dosing, about 250 to about 1600 ng/ml at about 2 hours post-dosing, about 150 to about 1500 ng/ml at about 4 hours post-dosing, about 90 to about 1200 ng/ml at 6 hours post-dosing; in another embodiment the formulation may yield a blood level of enoximone sulfoxide of 60 to about 800 ng/ml immediately after dosing, about 300 to about 1550 ng/ml at about 2 hours post-dosing, about 2000 to about 1350 ng/ml at about 4 hours post-dosing, about 11 to about 1125 ng/ml at 8 hours post-dosing; in another embodiment the formulation may yield a blood level of enoximone sulfoxide of 50 to about 800 ng/ml immediately after dosing, about 250 to about 1600 ng/ml at about 2 hours post-dosing, about 150 to about 1500 ng/ml at about 4 hours post-dosing, about 90 to about 1200 ng/ml at 8 hours post-dosing; in another embodiment the formulation may yield a blood level of enoximone sulfoxide of 60 to about 800 ng/ml immediately after dosing, about 300 to about 1550 ng/ml at about 2 hours post-dosing, about 200 to about 1350 ng/ml at about 4 hours post-dosing, about 11 to about 1125 ng/ml at 8 hours post-dosing;
a pharmaceutical formulation comprising about 66% Polysorbate-80 and 25 milligrams of enoximone, wherein the primary particle size of said enoximone is less than 10 μm on average, and wherein said formulation yields a blood level of enoximone of about 1 to about 150 ng/ml immediately after dosing, about 20 to about 400 ng/ml at about 2 hours post-dosing, about 15 to about 200 ng/ml at about 4 hours post-dosing, about 1 to about 150 ng/ml at about 8 hours post-dosing; or wherein the formulation yields a blood level profile of enoximone sulfoxide of about 40 to about 800 ng/ml immediately after dosing, about 80 to about 1500 ng/ml at about 2 hours post-dosing, about 80 to about 1500 ng/ml at about 4 hours post-dosing, about 40 to about 800 ng/ml at about 8 hours post-dosing; in another embodiment the formulation may yield a blood level of enoximone of 5 to about 160 ng/ml immediately after dosing, about 20 to about 400 ng/ml at about 2 hours post-dosing, about 15 to about 200 ng/ml at about 4 hours post-dosing, about 1 to about 150 ng/ml at 8 hours post-dosing; in another embodiment the formulation may yield a blood level of enoximone of 5 to about 160 ng/ml immediately after dosing, about 30 to about 500 ng/ml at about 2 hours post-dosing, about 20 to about 300 ng/ml at about 4 hours post-dosing, about 5 to about 200 ng/ml at 6 hours post-dosing; in another embodiment the formulation may yield a blood level of enoximone of 5 to about 160 ng/ml immediately after dosing, about 30 to about 500 ng/ml at about 2 hours post-dosing, about 20 to about 300 ng/ml at about 4 hours post-dosing, about 5 to about 200 ng/ml at 6 hours post-dosing; in another embodiment the formulation may yield a blood level of enoximone of 5 to about 150 ng/ml immediately after dosing, about 30 to about 400 ng/ml at about 2 hours post-dosing, about 20 to about 200 ng/ml at about 4 hours post-dosing, about 5 to about 150 ng/ml at 8 hours post-dosing; in another embodiment the formulation may yield a blood level of enoximone of 7 to about 149 ng/ml immediately after dosing, about 60 to about 385 ng/ml at about 2 hours post-dosing, about 40 to about 270 ng/ml at about 4 hours post-dosing, about 5 to about 150 ng/ml at 8 hours post-dosing; in another embodiment the formulation may yield a blood level of enoximone of 5 to about 150 ng/ml immediately after dosing, about 50 to about 390 ng/ml at about 2 hours post-dosing, about 40 to about 270 ng/ml at about 4 hours post-dosing, about 15 to about 170 ng/ml at 6 hours post-dosing; in another embodiment the formulation may yield a blood level of enoximone of 7 to about 150 ng/ml immediately after dosing, about 60 to about 385 ng/ml at about 2 hours post-dosing, about 40 to about 270 ng/ml at about 4 hours post-dosing, about 20 to about 170 ng/ml at 6 hours post-dosing; in another embodiment the formulation may yield a blood level of enoximone of 5 to about 160 ng/ml immediately after dosing, about 30 to about 500 ng/ml at about 2 hours post-dosing, about 20 to about 300 ng/ml at about 4 hours post-dosing, about 5 to about 200 ng/ml at 8 hours post-dosing; in another embodiment the formulation may yield a blood level of enoximone of 5 to about 150 ng/ml immediately after dosing, about 50 to about 390 ng/ml at about 2 hours post-dosing, about 40 to about 270 ng/ml at about 4 hours post-dosing, about 15 to about 170 ng/ml at 8 hours post-dosing; in another embodiment the formulation may yield a blood level of enoximone of 7 to about 150 ng/ml immediately after dosing, about 60 to about 385 ng/ml at about 2 hours post-dosing, about 40 to about 270 ng/ml at about 4 hours post-dosing, about 205 to about 170 ng/ml at 8 hours post-dosing; in another embodiment the formulation may yield a blood level of enoximone sulfoxide of 40 to about 800 ng/ml immediately after dosing, about 80 to about 1500 ng/ml at about 2 hours post-dosing, about 80 to about 1500 ng/ml at about 4 hours post-dosing, about 40 to about 800 ng/ml at 8 hours post-dosing; in another embodiment the formulation may yield a blood level of enoximone sulfoxide of 50 to about 900 ng/ml immediately after dosing, about 200 to about 1700 ng/ml at about 2 hours post-dosing, about 150 to about 1500 ng/ml at about 4 hours post-dosing, about 50 to about 900 ng/ml at 8 hours post-dosing; in another embodiment the formulation may yield a blood level of enoximone sulfoxide of 50 to about 900 ng/ml immediately after dosing, about 200 to about 1700 ng/ml at about 2 hours post-dosing, about 150 to about 1500 ng/ml at about 4 hours post-dosing, about 90 to about 1200 ng/ml at 6 hours post-dosing; in another embodiment the formulation may yield a blood level of enoximone sulfoxide of 50 to about 800 ng/ml immediately after dosing, about 200 to about 1500 ng/ml at about 2 hours post-dosing, about 150 to about 1500 ng/ml at about 4 hours post-dosing, about 50 to about 800 ng/ml at 8 hours post-dosing; in another embodiment the formulation may yield a blood level of enoximone sulfoxide of 60 to about 800 ng/ml immediately after dosing, about 300 to about 150 ng/ml at about 2 hours post-dosing, about 200 to about 1350 ng/ml at about 4 hours post-dosing, about 50 to about 800 ng/ml at 8 hours post-dosing; in another embodiment the formulation may yield a blood level of enoximone sulfoxide of 50 to about 800 ng/ml immediately after dosing, about 250 to about 1600 ng/ml at about 2 hours post-dosing, about 150 to about 1500 ng/ml at about 4 hours post-dosing, about 90 to about 1200 ng/ml at 6 hours post-dosing; in another embodiment the formulation may yield a blood level of enoximone sulfoxide of 60 to about 800 ng/ml immediately after dosing, about 300 to about 1550 ng/ml at about 2 hours post-dosing, about 200 to about 1350 ng/ml at about 4 hours post-dosing, about 11 to about 1125 ng/ml at 6 hours post-dosing; in another embodiment the formulation may yield a blood level of enoximone sulfoxide of 50 to about 900 ng/ml immediately after dosing, about 200 to about 1700 ng/ml at about 2 hours post-dosing, about 150 to about 1500 ng/ml at about 4 hours post-dosing, about 90 to about 1200 ng/ml at 8 hours post-dosing; in another embodiment the formulation may yield a blood level of enoximone sulfoxide of 50 to about 800 ng/ml immediately after dosing, about 250 to about 1600 ng/ml at about 2 hours post-dosing, about 150 to about 1500 ng/ml at about 4 hours post-dosing, about 90 to about 1200 ng/ml at 8 hours post-dosing; in another embodiment the formulation may yield a blood level of enoximone sulfoxide of 60 to about 800 ng/ml immediately after dosing, about 300 to about 1550 ng/ml at about 2 hours post-dosing, about 200 to about 1350 ng/ml at about 4 hours post-dosing, about 11 to about 1125 ng/ml at 8 hours post-dosing;
a pharmaceutical formulation comprising about 66% Polysorbate-80 and 50 milligrams of enoximone, wherein the primary particle size of said enoximone is less than 10 μm on average, and wherein said formulation yields a blood level of enoximone of about 1 to about 150 ng/ml immediately after dosing, about 20 to about 400 ng/ml at about 2 hours post-dosing, about 15 to about 200 ng/ml at about 4 hours post-dosing, about 1 to about 150 ng/ml at about 8 hours post-dosing; or wherein the formulation yields a blood level profile of enoximone sulfate of about 40 to about 800 ng/ml immediately after dosing, about 80 to about 1500 ng/ml at about 2 hours post-dosing, about 80 to about 1500 ng/ml at about 4 hours post-dosing, about 40 to about 800 ng/ml at about 8 hours post-dosing; in another embodiment the formulation may yield a blood level of enoximone of 5 to about 160 ng/ml immediately after dosing, about 20 to about 400 ng/ml at about 2 hours post-dosing, about 15 to about 200 ng/ml at about 4 hours post-dosing, about 1 to about 150 ng/ml at 8 hours post-dosing; in another embodiment the formulation may yield a blood level of enoximone of 5 to about 160 ng/ml immediately after dosing, about 30 to about 500 ng/ml at about 2 hours post-dosing, about 20 to about 300 ng/ml at about 4 hours post-dosing, about 5 to about 2000 ng/ml at 6 hours post-dosing; in another embodiment the formulation may yield a blood level of enoximone of 5 to about 160 ng/ml immediately after dosing, about 30 to about 500 ng/ml at about 2 hours post-dosing, about 20 to about 300 ng/ml at about 4 hours post-dosing, about 5 to about 2070 ng/ml at 6 hours post-dosing; in another embodiment the formulation may yield a blood level of enoximone of 5 to about 150 ng/ml immediately after dosing, about 30 to about 400 ng/ml at about 2 hours post-dosing, about 20 to about 200 ng/ml at about 4 hours post-dosing, about 5 to about 150 ng/ml at 8 hours post-dosing; in another embodiment the formulation may yield a blood level of enoximone of 7 to about 149 ng/ml immediately after dosing, about 60 to about 385 ng/ml at about 2 hours post-dosing, about 40 to about 270 ng/ml at about 4 hours post-dosing, about 5 to about 150 ng/ml at 8 hours post-dosing; in another embodiment the formulation may yield a blood level of enoximone of 5 to about 150 ng/ml immediately after dosing, about 50 to about 390 ng/ml at about 2 hours post-dosing, about 40 to about 270 ng/ml at about 4 hours post-dosing, about 15 to about 170 ng/ml at 6 hours post-dosing; in another embodiment the formulation may yield a blood level of enoximone of 7 to about 150 ng/ml immediately after dosing, about 60 to about 385 ng/ml at about 2 hours post-dosing, about 40 to about 270 ng/ml at about 4 hours post-dosing, about 20 to about 170 ng/ml at 6 hours post-dosing; in another embodiment the formulation may yield a blood level of enoximone of 5 to about 160 ng/ml immediately after dosing, about 30 to about 500 ng/ml at about 2 hours post-dosing, about 20 to about 300 ng/ml at about 4 hours post-dosing, about 5 to about 200 ng/ml at 8 hours post-dosing; in another embodiment the formulation may yield a blood level of enoximone of 7 to about 150 ng/ml immediately after dosing, about 60 to about 3850 ng/ml at about 2 hours post-dosing, about 40 to about 270 ng/ml at about 4 hours post-dosing, about 20 to about 170 ng/ml at 8 hours post-dosing; in another embodiment the formulation may yield a blood level of enoximone of 5 to about 150 ng/ml immediately after dosing, about 50 to about 390 ng/ml at about 2 hours post-dosing, about 40 to about 270 ng/ml at about 4 hours post-dosing, about 15 to about 170 ng/ml at 8 hours post-dosing; in another embodiment the formulation may yield a blood level of enoximone sulfoxide of 40 to about 800 ng/ml immediately after dosing, about 80 to about 1500 ng/ml at about 2 hours post-dosing, about 80 to about 1500 ng/ml at about 4 hours post-dosing, about 40 to about 800 ng/ml at 8 hours post-dosing; in another embodiment the formulation may yield a blood level of enoximone sulfoxide of 50 to about 900 ng/ml immediately after dosing, about 200 to about 1700 ng/ml at about 2 hours post-dosing, about 150 to about 1500 ng/ml at about 4 hours post-dosing, about 50 to about 900 ng/ml at 8 hours post-dosing; in another embodiment the formulation may yield a blood level of enoximone sulfoxide of 50 to about 900 ng/ml immediately after dosing, about 200 to about 1700 ng/ml at about 2 hours post-dosing, about 150 to about 1500 ng/ml at about 4 hours post-dosing, about 90 to about 1200 ng/ml at 6 hours post-dosing; in another embodiment the formulation may yield a blood level of enoximone sulfoxide of 50 to about 800 ng/ml immediately after dosing, about 200 to about 1500 ng/ml at about 2 hours post-dosing, about 150 to about 1500 ng/ml at about 4 hours post-dosing, about 50 to about 800 ng/ml at 8 hours post-dosing; in another embodiment the formulation may yield a blood level of enoximone sulfoxide of 60 to about 800 ng/ml immediately after dosing, about 300 to about 1550 ng/ml at about 2 hours post-dosing, about 200 to about 1350 ng/ml at about 4 hours post-dosing, about 50 to about 800 ng/ml at 8 hours post-dosing; in another embodiment the formulation may yield a blood level of enoximone sulfoxide of 50 to about 800 ng/ml immediately after dosing, about 250 to about 1600 ng/ml at about 2 hours post-dosing, about 150 to about 1500 ng/ml at about 4 hours post-dosing, about 90 to about 1200 ng/ml at 6 hours post-dosing; in another embodiment the formulation may yield a blood level of enoximone sulfoxide of 60 to about 800 ng/ml immediately after dosing, about 300 to about 1550 ng/ml at about 2 hours post-dosing, about 200 to about 1350 ng/ml at about 4 hours post-dosing, about 11 to about 1125 ng/ml at 6 hours post-dosing; in another embodiment the formulation may yield a blood level of enoximone sulfoxide of 50 to about 900 ng/ml immediately after dosing, about 200 to about 1700 ng/ml at about 2 hours post-dosing, about 150 to about 1500 ng/ml at about 4 hours post-dosing, about 90 to about 1200 ng/ml at 8 hours post-dosing; in another embodiment the formulation may yield a blood level of enoximone sulfoxide of 50 to about 800 ng/ml immediately after dosing, about 250 to about 1600 ng/ml at about 2 hours post-dosing, about 150 to about 1500 ng/ml at about 4 hours post-dosing, about 90 to about 1200 ng/ml at 8 hours post-dosing; in another embodiment the formulation may yield a blood level of enoximone sulfoxide of 60 to about 800 ng/ml immediately after dosing, about 300 to about 1550 ng/ml at about 2 hours post-dosing, about 200 to about 1350 ng/ml at about 4 hours post-dosing, about 11 to about 1125 ng/ml at 8 hours post-dosing.

Also provided are

In another embodiment, there is provided a method of treating heart failure comprising administering to a patient suffering from heart failure a therapeutic amount of a pharmaceutical formulation comprising enoximone and about 40-80% non-ionic surfactant by weight, wherein the primary particle size of said enoximone is less than 10 μm on average. The method may include or exclude administering to the patient a β-adrenergic receptor antagonist, and the method may be performed in the presence or absence of any other cardiovascular or cardiotonic drug. The enoximone formulation may be administered in a unit dosage of 25 mg or 50 mg three times per day. The non-ionic surfactant may comprise about 66% of the formulation. The non-ionic surfactant may be Polysorbate-80.

In yet another embodiment, there is provided a method of treating cardiac hypertrophy comprising administering to a patient suffering from cardiac hypertrophy a therapeutic amount of a pharmaceutical formulation comprising enoximone and about 40-80% non-ionic surfactant by weight, wherein the primary particle size of said enoximone is less than 10 μm on average. The method may exclude administering to the patient a β-adrenergic receptor antagonist. The enoximone formulation may be administered in a unit dosage of 25 mg or 50 mg three times per day. The non-ionic surfactant may comprise about 66% of the formulation. The non-ionic surfactant may be Polysorbate-80.

In still yet another embodiment, there is provided a method of weaning a cardiac hypertrophy patient from β-blockade comprising administering to the patient an effective amount of a pharmaceutical formulation comprising enoximone and about 40-80% non-ionic surfactant by weight, wherein the primary particle size of said enoximone is less than 10 μm on average. The enoximone formulation may be administered in a unit dosage of 25 mg or 50 mg three times per day. The non-ionic surfactant may comprise about 66% of the formulation. The non-ionic surfactant may be Polysorbate-80. The patient may further suffer from heart failure.

In additional embodiments, methods a provided for (a) decreasing incidence of death associated with cardiac hypertrophy and/or heart failure; (b) increasing time to death associated with cardiac hypertrophy and/or heart failure; (c) decreasing number of hospitalizations associated with cardiac hypertrophy and/or heart failure; and (d) increasing time to first hospitalization associated with cardiac hypertrophy and/or heart failure, each of the methods comprising administering to a patient suffering from heart failure a therapeutic amount of a pharmaceutical formulation comprising enoximone and about 40-80% non-ionic surfactant by weight, for example, where the enoximone formulation is administered in a unit dosage of 25 mg or 50 mg three times per day, and wherein the primary particle size of said enoximone is less than 10 μm on average.

As used herein, “about” means plus or minus 5% from the stated value.

As used herein, “a” or “an” may mean one or more. As used herein in the claim(s), when used in conjunction with the word “comprising,” the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DESCRIPTION OF ILLUSTRATIVE EMBODIMENTS

Heart failure is one of the leading causes of morbidity and mortality in the world. In the U.S. alone, estimates indicate that 3 million people are currently living with cardiomyopathy and another 400,000 are diagnosed on a yearly basis. Dilated cardiomyopathy (DCM), also referred to as “congestive cardiomyopathy,” is the most common form of the cardiomyopathies and has an estimated prevalence of nearly 40 per 100,000 individuals (Durand et al., 1995). Although there are other causes of DCM, familiar dilated cardiomyopathy has been indicated as representing approximately 20% of “idiopathic” DCM. Approximately half of the DCM cases are idiopathic, with the remainder being associated with known disease processes. For example, serious myocardial damage can result from certain drugs used in cancer chemotherapy (e.g., doxorubicin and daunoribucin), or from chronic alcohol abuse. Peripartum cardiomyopathy is another idiopathic form of DCM, as is disease associated with infectious sequelae. In sum, cardiomyopathies, including DCM, are significant public health problems.

Heart disease and its manifestations, including coronary artery disease, myocardial infarction, congestive heart failure and cardiac hypertrophy, clearly present a major health risk in the United States today. The cost to diagnose, treat and support patients suffering from these diseases is well into the billions of dollars. Two particularly severe manifestations of heart disease are myocardial infarction and cardiac hypertrophy. With respect to myocardial infarction, typically an acute thrombocytic coronary occlusion occurs in a coronary artery as a result of atherosclerosis and causes myocardial cell death. Because cardiomyocytes, the heart muscle cells, are terminally differentiated and generally incapable of cell division, they are generally replaced by scar tissue when they die during the course of an acute myocardial infarction. Scar tissue is not contractile, fails to contribute to cardiac function, and often plays a detrimental role in heart function by expanding during cardiac contraction, or by increasing the size and effective radius of the ventricle, for example, becoming hypertrophic.

With respect to cardiac hypertrophy, one theory regards this as a disease that resembles aberrant development and, as such, raises the question of whether developmental signals in the heart can contribute to hypertrophic disease. Cardiac hypertrophy is an adaptive response of the heart to virtually all forms of cardiac disease, including those arising from hypertension, mechanical load, myocardial infarction, cardiac arrhythmias, endocrine disorders, and genetic mutations in cardiac contractile protein genes. While the hypertrophic response is initially a compensatory mechanism that augments cardiac output, sustained hypertrophy can lead to DCM, heart failure, and sudden death. In the United States, approximately half a million individuals are diagnosed with heart failure each year, with a mortality rate approaching 50%.

The causes and effects of cardiac hypertrophy have been extensively documented, but the underlying molecular mechanisms have not been elucidated. Understanding these mechanisms is a major concern in the prevention and treatment of cardiac disease and will be crucial as a therapeutic modality in designing new drugs that specifically target cardiac hypertrophy and cardiac heart failure. As pathologic cardiac hypertrophy typically does not produce any symptoms until the cardiac damage is severe enough to produce heart failure, the symptoms of cardiomyopathy are those associated with heart failure. These symptoms include shortness of breath, fatigue with exertion, the inability to lie flat without becoming short of breath (orthopnea), paroxysmal nocturnal dyspnea, enlarged cardiac dimensions, and/or swelling in the lower legs. Patients also often present with increased blood pressure, extra heart sounds, cardiac murmurs, pulmonary and systemic emboli, chest pain, pulmonary congestion, and palpitations. In addition, DCM causes decreased ejection fractions (i.e., a measure of both intrinsic systolic function and remodeling). The disease is further characterized by ventricular dilation and grossly impaired systolic function due to diminished myocardial contractility, which results in dilated heart failure in many patients. Affected hearts also undergo cell/chamber remodeling as a result of the myocyte/myocardial dysfunction, which contributes to the “DCM phenotype.” As the disease progresses so do the symptoms. Patients with DCM also have a greatly increased incidence of life-threatening arrhythmias, including ventricular tachycardia and ventricular fibrillation. In these patients, an episode of syncope (dizziness) is regarded as a harbinger of sudden death.

Enoximone, in an i.v. formulation (Perfan®), has been used to treat congestive heart failure and to treat patients in cardiac post-surgery or transplant settings. It is a member of a unique chemical class of drugs called imidazolone derivatives and possesses both positive inotropic and vasodilating properties. These dual actions are evidenced clinically by increased contractility plus reduced preload and afterload, resulting in increased cardiac output, with little or no effect on myocardial oxygen consumption. The molecular basis for these effects is the apparent inhibitory action of enoximone on Type IV phosphodiesterase, which results in an increase in intracellular levels of cAMP and the consequent inotropic effect. Unfortunately, the i.v. therapy typically requires participation of trained medical personnel, often in a hospital setting. Patient compliance also becomes an issue on self-medication. Therefore, oral formulations of enoximone are desirable. Previous attempts to utilize oral enoximone resulted in the cancellation of clinical trials due to increased lethality and severe adverse events or side effects at the doses chosen (Om and Hess, 1993). As such, the present invention provides specific lower dose oral formulations with particular pharmacologic effects that overcome the limitations of the prior art, and further the present invention provides for enoximone formulations wherein the particles sizes of enoximone are uniformly less than 10 microns, a significant departure from and improvement over the prior art.

I. Heart Failure And Hypertrophy

Heart disease and its manifestations, including coronary artery disease, myocardial infarction, congestive heart failure and cardiac hypertrophy, clearly presents a major health risk in the United States today. The cost to diagnose, treat and support patients suffering from these diseases is well into the billions of dollars. One particularly severe manifestations of heart disease is cardiac hypertrophy. Regarding hypertrophy, one theory regards this as a disease that resembles aberrant development and, as such, raises the question of whether developmental signals in the heart can contribute to hypertrophic disease. Cardiac hypertrophy is an adaptive response of the heart to virtually all forms of cardiac disease, including those arising from hypertension, mechanical load, myocardial infarction, cardiac arrhythmias, endocrine disorders, and genetic mutations in cardiac contractile protein genes. While the hypertrophic response is initially a compensatory mechanism that augments cardiac output, sustained hypertrophy can lead to DCM, heart failure, and sudden death. In the United States, approximately half a million individuals are diagnosed with heart failure each year, with a mortality rate approaching 50%.

The causes and effects of cardiac hypertrophy have been extensively documented, but the underlying molecular mechanisms have not been fully elucidated. Understanding these mechanisms is a major concern in the prevention and treatment of cardiac disease and will be crucial as a therapeutic modality in designing new drugs that specifically target cardiac hypertrophy and cardiac heart failure. The symptoms of cardiac hypertrophy initially mimic those of heart failure and may include shortness of breath, fatigue with exertion, the inability to lie flat without becoming short of breath (orthopnea), paroxysmal nocturnal dyspnea, enlarged cardiac dimensions, and/or swelling in the lower legs. Patients also often present with increased blood pressure, extra heart sounds, cardiac murmurs, pulmonary and systemic emboli, chest pain, pulmonary congestion, and palpitations. In addition, DCM causes decreased ejection fractions (i.e., a measure of both intrinsic systolic function and remodeling). The disease is further characterized by ventricular dilation and grossly impaired systolic function due to diminished myocardial contractility, which results in dilated heart failure in many patients. Affected hearts also undergo cell/chamber remodeling as a result of the myocyte/myocardial dysfunction, which contributes to the “DCM phenotype.” As the disease progresses so do the symptoms. Patients with DCM also have a greatly increased incidence of life-threatening arrhythmias, including ventricular tachycardia and ventricular fibrillation. In these patients, an episode of syncope (dizziness) is regarded as a harbinger of sudden death.

Diagnosis of hypertrophy typically depends upon the demonstration of enlarged heart chambers, particularly enlarged ventricles. Enlargement is commonly observable on chest X-rays, but is more accurately assessed using echocardiograms. DCM is often difficult to distinguish from acute myocarditis, valvular heart disease, coronary artery disease, and hypertensive heart disease. Once the diagnosis of dilated cardiomyopathy is made, every effort is made to identify and treat potentially reversible causes and prevent further heart damage. For example, coronary artery disease and valvular heart disease must be ruled out. Anemia, abnormal tachycardias, nutritional deficiencies, alcoholism, thyroid disease and/or other problems need to be addressed and controlled.

As mentioned above, treatment with pharmacological agents still represents the primary mechanism for reducing or eliminating the manifestations of heart failure. Diuretics constitute the first line of treatment for mild-to-moderate heart failure. Unfortunately, many of the commonly used diuretics (e.g., the thiazides) have numerous adverse effects. For example, certain diuretics may increase serum cholesterol and triglycerides. Moreover, diuretics are generally ineffective for patients suffering from severe heart failure.

If diuretics are ineffective, vasodilatory agents may be used; the angiotensin converting (ACE) inhibitors (e.g., enalopril and lisinopril) not only provide symptomatic relief, they also have been reported to decrease mortality (Young et al., 1989). Again, however, the ACE inhibitors are associated with adverse effects that result in their being contraindicated in patients with certain disease states (e.g., renal artery stenosis). Similarly, inotropic agent therapy (i.e., a drug that improves cardiac output by increasing the force of myocardial muscle contraction) has previously been associated with a panoply of adverse reactions, including gastrointestinal problems and central nervous system dysfunction.

Thus, the currently used pharmacological agents have severe shortcomings in particular patient pop,ulations. The availability of new, safe and effective agents would undoubtedly benefit patients who either cannot use the pharmacological modalities presently available, or who do not receive adequate relief from those modalities.

II. Enoximone

Enoximone (1,3-Dihydro-4-methyl-5-[4-(methylthio)benzoyl]-2H-imidazol-2-one) is a small organic molecule that exhibits highly selective inhibition of type-III phosphodiesterase, or PDE-III, an enzyme that is present in the heart and plays an important regulatory role in cardiac function. PDE-III inhibitors block the action of this enzyme, increasing the force of contraction of the heart, thereby increasing cardiac output. Compounds that increase the force of contraction of the heart, like enoximone, are referred to as positive inotropes. Enoximone also causes vasodilation, an increase in the diameter of blood vessels, through its effects on smooth muscle cells that surround blood vessels, which results in lower pressure against which the heart must pump. Positive inotropy and vasodilation can both be therapeutically useful in the treatment of heart failure. Enoximone is described in detail in U.S. Pat. No. 4,505,635, which is hereby incorporated by reference. Enoximone is further described in EP Patent 326103, a formulations patent, which is also hereby incorporated by reference. The inventors herein describe an improvement over the '103 EP Patent in that it has been subsequently discovered that particle size is important for bioavailability, and achieving uniformity of particle size to less than 10 microns significantly enhances bioavailability. Prior enoximone oral formulations relied on a triple mill press to relieve agglomeration problems (EP 326103). This process was found to yield particles sizes ranging from 25 to 250 microns, with a fairly uniform distribution centered around 100 microns (data unpublished). Limited solubility of enoximone in the physiological pH range was later found to reduce bioavailability of the drug product and the inventors discovered that for therapeutic bioavailability the particles had to be uniformly less than 10 microns (data unpublished). The present invention, therefore, is drawn to enoximone that is micronized in a way that yields uniformity of particle size to less than 10 microns. As such, the present invention represents a significant advance and improvement over formulations presented in the '103 EP Patent or the '635 U.S. patent.

Perfan I.V. is an intravenous formulation of enoximone that is currently marketed in eight European countries. Clinical studies supporting the use of Perfan I.V. were completed in the late 1980s, and the drug was first approved in Europe in 1989. Perfan I.V. is used in a hospital setting to treat patients with acute decompensated heart failure (Classes III and IV) and to wean patients from cardiopulmonary bypass following open-heart surgery. This treatment, along with the use of powerful intravenous diuretics, vasodilators, all of which serve to increase the efficiency of the circulatory system and provide symptomatic relief. After stabilization and discharge from the hospital, patients often decompensate again within months and must be readmitted to the hospital for another round of intravenous treatment. As their disease progresses, the frequency of decompensation and hospitalization increases until patients must be maintained on continuous or intermittent treatment with these intravenous agents, which is both confining and costly.

Patients with advanced chronic heart failure can benefit greatly from the chronic use of an oral inotropic agent that would provide the desired symptomatic relief to the patients and reduce the frequency of hospitalizations by delaying additional episodes of acute decompensated heart failure. An oral product with these characteristics could also wean patients with severe heart failure who are currently dependent on intravenous inotropic therapy from those agents and allow them the opportunity to leave the hospital and return to a more normal daily life. Such an agent would decrease the overall costs associated with the treatment of heart failure.

Four Phase III trials of low-dose enoximone capsules are currently underway for patients with advanced chronic heart failure in an effort to overcome the problems seen with a series of clinical trials using higher doses of enoximone. In the 1980s, Merrell Dow (now part of Aventis) conducted clinical evaluation of enoximone capsules for the treatment of chronic heart failure. Enoximone capsules were evaluated in approximately 5,000 patients with chronic heart failure in multiple Phase I and Phase II clinical trials conducted in the United States, Europe and Japan. The drug was initially tested at doses now considered high—100 to 300 milligrams administered three times a day. At these high doses, patients treated with enoximone capsules demonstrated clinically significant increases in quality of life scores and maximal exercise capacity. However, in one Phase II placebo-controlled trial involving 151 patients administered enoximone capsules at doses of 100 milligrams or placebo capsules three times a day, there was a statistically significant increase in the mortality rate in the group of patients receiving enoximone capsules compared to the group receiving placebo capsules: 36% of the patients treated with enoximone capsules died during the trial versus 23% of the patients treated with placebo.

Dr. Michael Bristow (Univ. of Colorado Health Science Center) made the unexpected observation that enoximone capsules administered at lower doses appeared to retain efficacy without increasing mortality Bristow (1994). Subsequently, a series of Phase II clinical trials have reported that (a) enoximone capsules administered at doses of 25 and 50 mg three times a day increased maximal exercise capacity with no apparent increase in mortality in patients with Class II and III chronic heart failure after 12 weeks of treatment (two placebo-controlled trials involving a total of 273 patients); (b) enoximone capsules administered at doses of 25 to 75 mg three times a day extended the survival times of patients with Class IV chronic heart failure awaiting a heart transplant (186-patient open-label, parallel-control trial); (c) and enoximone capsules administered at doses of 25 and 50 mg three times a day enabled patients with Class IV chronic heart failure, and otherwise too weak to tolerate beta-blockers, to receive and benefit from beta-blocker therapy. These benefits included a significant reduction in the severity of their chronic heart failure symptoms and hospitalization events (30-patient, open-label trial). In addition, Dr. Bristow has conducted a series of open-label trials of enoximone capsules involving over 200 patients to gather additional clinical data. However, the reported studies were preliminary in nature and merely constitute an experimental use of enoximone.

Another clinical trial conducted by Dr. Bristow gave rise to U.S. Pat. No. 5,998,458. This patent claims the use of positive inotropic therapy in combination with β blockade in a specific manner, including oral enoximone formulations. However, again, the described clinical trials were not sufficient to constitute anything more than an experimental use.

In June of 2000, Myogen, Inc. initiated a Phase III program to evaluate the safety and efficacy of enoximone capsules for the long-term treatment of patients with advanced chronic heart failure. In these studies, enoximone capsules are being used in addition to standard therapies, including diuretics, ACE inhibitors and beta-blockers. The Phase III program includes four trials designed to collectively demonstrate that enoximone capsules at doses of 25 or 50 mg administered three times a day are effective in reducing hospitalizations, improving symptoms of chronic heart failure, improving quality of life and reducing the need for intravenous inotropic therapy.

EMOTE was a randomized, double-blind, placebo-controlled Phase III trial of approximately 200 patients with the most advanced stage of chronic heart failure, and who are dependent on intravenous inotrope therapy. The trial was designed to evaluate the use of enoximone capsules to wean patients off of intravenous inotrope therapy. Patients received 26 weeks of treatment. This trial was conducted in the United States. Preliminary results of EMOTE were reported in March 2004: Analysis of the primary endpoint, wean success at 30 days, demonstrated a wean success rate of 61% in the enoximone-treated group and 51% in the placebo-treated group. This difference did not reach statistical significance (p=0.171). The key secondary endpoints, which also evaluated wean from i.v. inotrope therapy, but over time rather than at a fixed 30-day time point, were achieved, demonstrating a therapeutic benefit. The safety results demonstrated no statistical difference in serious adverse events or mortality between the groups receiving placebo or enoximone capsules. A total of 38/101 patients died in the enoximone treated group compared to 31/100 patients in the placebo treated group (p=0.335) (www.myogen.com/pipeline/enoximone.php, last visited Dec. 9, 2004).

The first secondary endpoint, time to death or re-initiation of i.v. inotrope therapy, a measure of wean success over time, demonstrated benefit in favor of enoximone versus placebo for the 60-day (p=0.012), 75-day (p=0.016) and 90-day (p=0.041) periods, but not for the 182-day (p=0.188) period. Mean total days on i.v. inotrope therapy over the course of the study, another secondary endpoint, were 28 days in the enoximone-treated group as compared to 49 days in the placebo-treated group (p=0.049) (www.myogen.com/pipeline/enoximone.php, last visited Dec. 9, 2004).

ESSENTIAL I is a randomized, double-blind, placebo-controlled pivotal Phase III trial of approximately 900 patients with Class III and IV chronic heart failure that are being treated with beta-blockers and other therapies according to current guidelines. The trial will track the time from randomization to cardiovascular hospitalization or death for each patient as the primary endpoint. On average, patients will receive treatment for at least 12 months. This trial is being conducted in North and South America. Patient enrollment began in February 2002.

ESSENTIAL II is a Phase III trial identical in design and size to ESSENTIAL I. This trial is being conducted in Western and Eastern Europe. Patient enrollment began in April 2002.

EMPOWER is a randomized, double-blind, placebo-controlled Phase III trial of approximately 175 patients with Class III and IV chronic heart failure. Patients will be treated for 26 to 36 weeks with either (i) placebo, (ii) extended release metoprolol, a frequently prescribed beta-blocker, or (iii) extended release metoprolol in combination with enoximone capsules. The primary objective of this study is to determine whether enoximone capsules can increase the tolerability to metoprolol in patients previously shown to be intolerant to beta-blocker treatment. Patient enrollment began in September 2003.

A. Synthesis

Enoximone may be prepared according to the following method. A solution of 25.0 g of 4-(methylthio)-benzoic acid and 22 ml of thionyl chloride in 50 ml of benzene is refluxed for 4 hrs. Excess reagent and solvent is evaporated and the residue is azeotroped 3 times with benzene to remove all thionyl chloride. The residue is added dropwise to a mixture of 11.8 g of 1,3-dihydro-4-methyl-2H-imidazol-2-one, 40.0 g of anhydrous aluminum chloride and 100 ml of nitrobenzene. The resulting mixture is stirred at 60°-65° C. for 5 hrs, poured on ice and the precipitate that forms is collected, washed with ethyl ether and water, and recrystallized from isopropanol-water to give the title compound. M.P. 255°-258° C. (dec.).

B. Micronized Forms

In many drug manufacturing, milling and micronizing machines pulverize substances into extremely fine particles, and thus reduce bulk chemicals to the required size for pharmaceutical formulation. Particles may also be micronized by chemical or temperature controlled processes. The primary benefit to micronizing is the increase in solubility/bioavailability due to the increase in surface area. These finished chemicals are combined and processed further in mixing machines. The mixed ingredients may then be mechanically capsulated, pressed into tablets, or made into solutions. As used herein, the term “micronizing” may be considered to refer to the processes of making uniform particle size of a drug, wherein the size desired may be 10 microns or less, and wherein said process may be mechanical, chemical, temperature or pH controlled, or any other commonly known process familiar to one of skill in the art.

Optimization and control of micronizing processes, particularly relating to particle size, are becoming ever more important in the development of pharmaceuticals. Air jet micronization is a well proven technique that consistently produces particles in the 1-30 micron range. Micron Technologies and Jet Pharma are contract micronizers. The primary advantages of air jet micronizers are that particle reduction occurs via particle to particle collisions, with limited reduction from metal to product contact, and no generation of heat. Other advantages include no moving parts and easy to clean surfaces.

The original principles of jet milling are simple. The powder particles are fed into the flat cylindrical milling chamber tangentially through a venturi system by pressurized air or nitrogen. The particles are accelerated in a spiral movement inside the milling chamber by a number of nozzles placed around the periphery of the chamber. The micronizing effect takes place by the collision between the incoming particles and those already accelerated into the spiral path. While centrifugal force retains the larger particles at the periphery of the milling chamber, the smaller particles exit with the exhaust air from the centre of the chamber. The particle size distribution is controlled by adjusting a number of parameters, two of the main ones being pressure and feed rate.

U.S. Pat. Nos. 6,645,466, 6,623,760, 6,555,135, hereby incorporated by reference, describe other micronization procedures.

III. Non-Ionic Surfactants

The non-ionic surfactant of the present invention may comprise any one of a number of different agents. Included are sorbitan esters (sorbitan monolaurate, sorbitan monooleate, sorbitan monopalmitate, sorbitan monostearate, sorbitan sesquioleate, sorbitan trioleate), ethoxylated sorbitan esters (polyethoxyethylene sorbitan fatty acid esters, polysorbates, Tween™), ethoxylated (polyethoxyethylene) fatty alcohols, ethoxylated (polyethoxyethylene) fatty acids, poloxamers (Pluronic™), polyglycolized glycerides (Labrasol™, Labrafil™, Gelucires™), polyoxyethylene alkyl ethers (Brij™), polyoxyethylene castor oil derivatives (Cremphor™), vitamin E TPGS (tocopheryl polyethylene glycol succinate), glyceryl monooleates, polyvinyl alcohols, and olyoxyethylene alkyl ethers. See Handbook of Pharmaceutical Excipients (2000); Handbook of Industrial Surfactants (2000); U.S. Pat. Nos. 6,254,885 and 6,596,308. The surfactant will be present in amounts exceding 40%, but may be greater than 45%, 50%, 55%, 60%, 65%, 66%, 67%, 68%, 69%, 70%, 75%, but no more than 80%.

IV. Methods of Treating Heart Failure And Cardiac Hypertrophy

A. Therapeutic Regimens For Heart Failure And Hypertrophy

Heart failure of some forms may curable and these are dealt with by treating the primary disease, such as anemia or thyrotoxicosis. Also curable are forms caused by anatomical problems, such as a heart valve defect. These defects can be surgically corrected. However, for the most common forms of heart failure—those due to damaged heart muscle—no known cure exists. Treating the symptoms of these diseases helps, and some treatments of the disease have been successful. The treatments attempt to improve patients' quality of life and length of survival through lifestyle change and drug therapy. Patients can minimize the effects of heart failure by controlling the risk factors for heart disease, but even with lifestyle changes, most heart failure patients must take medication, many of whom receive two or more drugs.

Several types of drugs have proven useful in the treatment of heart failure: Diuretics help reduce the amount of fluid in the body and are useful for patients with fluid retention and hypertension; and digitalis can be used to increase the force of the heart's contractions, helping to improve circulation. Results of recent studies have placed more emphasis on the use of ACE inhibitors (Manoria and Manoria, 2003). Several large studies have indicated that ACE inhibitors improve survival among heart failure patients and may slow, or perhaps even prevent, the loss of heart pumping activity (for a review see De Feo et al., 2003; DiBianco, 2003).

Patients who cannot take ACE inhibitors may get a nitrate and/or a drug called hydralazine, each of which helps relax tension in blood vessels to improve blood flow (Ahmed, 2003).

Heart failure is almost always life-threatening. When drug therapy and lifestyle changes fail to control its symptoms, a heart transplant may be the only treatment option. However, candidates for transplantation often have to wait months or even years before a suitable donor heart is found. Recent studies indicate that some transplant candidates improve during this waiting period through drug treatment and other therapy, and can be removed from the transplant list (Conte et al., 1998).

Transplant candidates who do not improve sometimes need mechanical pumps, which are attached to the heart. Called left ventricular assist devices (LVADs), the machines take over part or virtually all of the heart's blood-pumping activity. However, current LVADs are not permanent solutions for heart failure but are considered bridges to transplantation.

As a final alternative, there is an experimental surgical procedure for severe heart failure available called cardiomyoplasty (Dumcius et al., 2003) This procedure involves detaching one end of a muscle in the back, wrapping it around the heart, and then suturing the muscle to the heart. An implanted electric stimulator causes the back muscle to contract, pumping blood from the heart. To date, none of these treatments have been shown to cure heart failure, but can at least improve quality of life and extend life for those suffering this disease.

As with heart failure, there are no known cures to hypertrophy. Current medical management of cardiac hypertrophy, in the setting of a cardiovascular disorder includes the use of at least two types of drugs: inhibitors of the rennin-angiotensoin system, and β-adrenergic blocking agents (Bristow, 1999). Therapeutic agents to treat pathologic hypertrophy in the setting of heart failure include angiotensin II converting enzyme (ACE) inhibitors and β-adrenergic receptor blocking agents (Eichhorn and Bristow, 1996). Other pharmaceutical agents that have been disclosed for treatment of cardiac hypertrophy include angiotensin II receptor antagonists (U.S. Pat. No. 5,604,251) and neuropeptide Y antagonists (WO 98/33791).

Non-pharmacological treatment is primarily used as an adjunct to pharmacological treatment. One means of non-pharmacological treatment involves reducing the sodium in the diet. In addition, non-pharmacological treatment also entails the elimination of certain precipitating drugs, including negative inotropic agents (e.g., certain calcium channel blockers and antiarrhythmic drugs like disopyramide), cardiotoxins (e.g:, amphetamines), and plasma volume expanders (e.g., nonsteroidal anti-inflammatory agents and glucocorticoids).

As can be seen from the discussion above, there is a great need for a successful treatment approach to heart failure and hypertrophy. In one embodiment of the present invention, methods for the treatment of cardiac hypertrophy or heart failure utilizing enoximone formulations of the present invention. For the purposes of the present application, treatment comprises reducing one or more of the symptoms of heart failure or cardiac hypertrophy, such as reduced exercise capacity, reduced blood ejection volume, increased left ventricular end diastolic pressure, increased pulmonary capillary wedge pressure, reduced cardiac output, cardiac index, increased pulmonary artery pressures, increased left ventricular end systolic and diastolic dimensions, and increased left ventricular wall stress, wall tension and wall thickness-same for right ventricle. In addition, use of inhibitors of enoximone may prevent cardiac hypertrophy and its associated symptoms from arising.

B. Enoximone Regimens

The proposed enoximone regimen will comprise three daily administrations of 25 mg or 50 mg oral dosage form of enoximone. Changes in various hemodynamic parameters will be recorded before enoximone administration and at periodic times (e.g., 2, 4, 6, 8, 10, 12, 24 hrs) following administration. Typical parameters include heart rate, blood pressure, and cardiac output. Other parameters to be measured include pulmonary arterial wedge pressure, pulmonary aterial pressure, right atrial pressure, diastolic pulmonary aterial pressure.

Initial treatment will normally begin only after cessation of digitalises, diuretics, beta-stimulants and vasodilators (normally stopped 12, 24 or 48 hr before). Combinations therapies (see below) may be commenced at such time thereafter as considered appropriate by the treating physician.

C. Combined Therapy

In another embodiment, it is envisioned to use enoximone in combination with other therapeutic modalities. Thus, in addition to the therapies described above, one may also provide to the patient more “standard” pharmaceutical cardiac therapies. Examples of other therapies include, without limitation, so-called “beta blockers,” anti-hypertensives, cardiotonics, anti-thrombotics, vasodilators, hormone antagonists, other inotropes, diuretics, endothelin antagonists, calcium channel blockers, phosphodiesterase inhibitors, ACE inhibitors, angiotensin type 2 antagonists and cytokine blockers/inhibitors, and HDAC inhibitors.

Combinations may be achieved by contacting cardiac cells with a single composition or pharmacological formulation that includes both agents, or by contacting the cell with two distinct compositions or formulations, at the same time, wherein one composition includes the expression construct and the other includes the agent. Alternatively, the therapy using enoximone may precede or follow administration of the other agent(s) by intervals ranging from minutes to weeks. In embodiments where the other agent and enoximone are applied separately to the cell, one would generally ensure that a significant period of time did not expire between the time of each delivery, such that the agent and enoximone would still be able to exert an advantageously combined effect on the cell. In such instances, it is contemplated that one would typically contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either enoximone or the other agent will be desired. In this regard, various combinations may be employed. By way of illustration, where enoximone is “A” and the other agent is “B,” the following permutations based on 3 and 4 total administrations are exemplary:
A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B
Other combinations are likewise contemplated.

D. Adjunct Therapeutic Agents For Combination Therapy

Pharmacological therapeutic agents and methods of administration, dosages, etc., are well known to those of skill in the art (see for example, the “Physicians Desk Reference,” Goodman and Gilman's “The Pharmacological Basis of Therapeutics,” “Remington's Pharmaceutical Sciences,” and “The Merck Index, Thirteenth Edition,” incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject, and such invidual determinations are within the skill of those of ordinary skill in the art.

Non-limiting examples of a pharmacological therapeutic agent that may be used in the present invention include an antihyperlipoproteinemic agent, an antiarteriosclerotic agent, an antithrombotic/fibrinolytic agent, a blood coagulant, an antiarrhythmic agent, an antihypertensive agent, a vasopressor, a treatment agent for congestive heart failure, an antianginal agent, an antibacterial agent or a combination thereof.

In addition, it should be noted that any of the following may be used to develop new sets of cardiac therapy target genes as β-blockers were used in the present examples (see below). While it is expected that many of these genes may overlap, new gene targets likely can be developed.
1. Antihyperlipoproteinemics

In certain embodiments, administration of an agent that lowers the concentration of one of more blood lipids and/or lipoproteins, known herein as an “antihyperlipoproteinemic,” may be combined with a cardiovascular therapy according to the present invention, particularly in treatment of athersclerosis and thickenings or blockages of vascular tissues. In certain aspects, an antihyperlipoproteinemic agent may comprise an aryloxyalkanoic/fibric acid derivative, a resin/bile acid sequesterant, a HMG CoA reductase inhibitor, a nicotinic acid derivative, a thyroid hormone or thyroid hormone analog, a miscellaneous agent or a combination thereof.
a. Aryloxyalkanoic Acid/Fibric Acid Derivatives

Non-limiting examples of aryloxyalkanoic/fibric acid derivatives include beclobrate, enzafibrate, binifibrate, ciprofibrate, clinofibrate, clofibrate (atromide-S), clofibric acid, etofibrate, fenofibrate, gemfibrozil (lobid), nicofibrate, pirifibrate, ronifibrate, simfibrate and theofibrate.
b. Resins/Bile Acid Sequesterants

Non-limiting examples of resins/bile acid sequesterants include cholestyramine (cholybar, questran), colestipol (colestid) and polidexide.
c. HMG CoA Reductase Inhibitors

Non-limiting examples of HMG CoA reductase inhibitors include lovastatin (mevacor), pravastatin (pravochol) or simvastatin (zocor).
d. Nicotinic Acid Derivatives

Non-limiting examples of nicotinic acid derivatives include nicotinate, acepimox, niceritrol, nicoclonate, nicomol and oxiniacic acid.
e. Thryroid Hormones and Analogs

Non-limiting examples of thyroid hormones and analogs thereof include etoroxate, thyropropic acid and thyroxine.
f. Miscellaneous Antihyperlipoproteinemics

Non-limiting examples of miscellaneous antihyperlipoproteinemics include acifran, azacosterol, benfluorex, b-benzalbutyramide, carnitine, chondroitin sulfate, clomestrone, detaxtran, dextran sulfate sodium, 5,8,11,14,17-eicosapentaenoic acid, eritadenine, furazabol, meglutol, melinamide, mytatrienediol, ornithine, g-oryzanol, pantethine, pentaerythritol tetraacetate, a-phenylbutyramide, pirozadil, probucol (lorelco), b-sitosterol, sultosilic acid-piperazine salt, tiadenol, triparanol and xenbucin.
2. Antiarteriosclerotics

Non-limiting examples of an antiarteriosclerotic include pyridinol carbamate.
3. Antithrombotic/Fibrinolytic Agents

In certain embodiments, administration of an agent that aids in the removal or prevention of blood clots may be combined with administration of a modulator, particularly in treatment of athersclerosis and vasculature (e.g., arterial) blockages. Non-limiting examples of antithrombotic and/or fibrinolytic agents include anticoagulants, anticoagulant antagonists, antiplatelet agents, thrombolytic agents, thrombolytic agent antagonists or combinations thereof.

In certain aspects, antithrombotic agents that can be administered orally, such as, for example, aspirin and wafarin (coumadin), are preferred.
a. Anticoagulants

A non-limiting example of an anticoagulant include acenocoumarol, ancrod, anisindione, bromindione, clorindione, coumetarol, cyclocumarol, dextran sulfate sodium, dicumarol, diphenadione, ethyl biscoumacetate, ethylidene dicoumarol, fluindione, heparin, hirudin, lyapolate sodium, oxazidione, pentosan polysulfate, phenindione, phenprocoumon, phosvitin, picotamide, tioclomarol and warfarin.
b. Antiplatelet Agents

Non-limiting examples of antiplatelet agents include aspirin, a dextran, dipyridamole (persantin), heparin, sulfinpyranone (anturane) and ticlopidine (ticlid).

c. Thrombolytic Agents

Non-limiting examples of thrombolytic agents include tissue plasminogen activator (activase), plasmin, pro-urokinase, urokinase (abbokinase) streptokinase (streptase), anistreplase/APSAC (eminase).
4. Blood Coagulants

In certain embodiments wherein a patient is suffering from a hemhorrage or an increased likelyhood of hemhorraging, an agent that may enhance blood coagulation may be used. Non-limiting examples of a blood coagulation promoting agent include thrombolytic agent antagonists and anticoagulant antagonists.
a. Anticoagulant Antagonists

Non-limiting examples of anticoagulant antagonists include protamine and vitamine K1.
b. Thrombolytic Agent Antagonists And Antithrombotics

Non-limiting examples of thrombolytic agent antagonists include amiocaproic acid (amicar) and tranexarnic acid (amstat). Non-limiting examples of antithrombotics include anagrelide, argatroban, cilstazol, daltroban, defibrotide, enoxaparin, fraxiparine, indobufen, lamoparan, ozagrel, picotamide, plafibride, tedelparin, ticlopidine and triflusal.
5. Antiarrhythmic Agents

Non-limiting examples of antiarrhythmic agents include Class I antiarrhythmic agents (sodium channel blockers), Class II antiarrhythmic agents (beta-adrenergic blockers), Class II antiarrhythmic agents (repolarization prolonging drugs), Class IV antiarrhythmic agents (calcium channel blockers) and miscellaneous antiarrhythmic agents.
a. Sodium Channel Blockers

Non-limiting examples of sodium channel blockers include Class IA, Class IB and Class IC antiarrhythmic agents. Non-limiting examples of Class IA antiarrhythmic agents include disppyramide (norpace), procainamide (pronestyl) and quinidine (quinidex). Non-limiting examples of Class IB antiarrhythmic agents include lidocaine (xylocaine), tocainide (tonocard) and mexiletine (mexitil). Non-limiting examples of Class IC antiarrhythmic agents include encainide (enkaid) and flecainide (tambocor).
b. Beta Blockers

Non-limiting examples of a beta blocker, otherwise known as a b-adrenergic blocker, a b-adrenergic antagonist or a Class II antiarrhythmic agent, include acebutolol (sectral), alprenolol, amosulalol, arotinolol, atenolol, befunolol, betaxolol, bevantolol, bisoprolol, bopindolol, bucumolol, bufetolol, bufuralol, bunitrolol, bupranolol, butidrine hydrochloride, butofilolol, carazolol, carteolol, carvedilol, celiprolol, cetamolol, cloranolol, dilevalol, epanolol, esmolol (brevibloc), indenolol, labetalol, levobunolol, mepindolol, metipranolol, metoprolol, moprolol, nadolol, nadoxolol, nifenalol, nipradilol, oxprenolol, penbutolol, pindolol, practolol, pronethalol, propanolol (inderal), sotalol (betapace), sulfinalol, talinolol, tertatolol, timolol, toliprolol and xibinolol. In certain aspects, the beta blocker comprises an aryloxypropanolamine derivative. Non-limiting examples of aryloxypropanolamine derivatives include acebutolol, alprenolol, arotinolol, atenolol, betaxolol, bevantolol, bisoprolol, bopindolol, bunitrolol, butofilolol, carazolol, carteolol, carvedilol, celiprolol, cetamolol, epanolol, indenolol, mepindolol, metipranolol, metoprolol, moprolol, nadolol, nipradilol, oxprenolol, penbutolol, pindolol, propanolol, talinolol, tertatolol, timolol and toliprolol.
c. Repolarization Prolonging Agents

Non-limiting examples of an agent that prolong repolarization, also known as a Class III antiarrhythmic agent, include amiodarone (cordarone) and sotalol (betapace).
d. Calcium Channel Blockers/Antagonist

Non-limiting examples of a calcium channel blocker, otherwise known as a Class IV antiarrhythmic agent, include an arylalkylamine (e.g., bepridile, diltiazem, fendiline, gallopamil, prenylamine, terodiline, verapamil), a dihydropyridine derivative (felodipine, isradipine, nicardipine, nifedipine, nimodipine, nisoldipine, nitrendipine) a piperazinde derivative (e.g., cinnarizine, flunarizine, lidoflazine) or a micellaneous calcium channel blocker such as bencyclane, etafenone, magnesium, mibefradil or perhexiline. In certain embodiments a calcium channel blocker comprises a long-acting dihydropyridine (amlodipine) calcium antagonist.
e. Miscellaneous Antiarrhythmic Agents
Non-limiting examples of miscellaneous antiarrhymic agents include adenosine (adenocard), digoxin (lanoxin), acecainide, ajmaline, amoproxan, aprindine, bretylium tosylate, bunaftine, butobendine, capobenic acid, cifenline, disopyranide, hydroquinidine, indecainide, ipatropium bromide, lidocaine, lorajmine, lorcainide, meobentine, moricizine, pirmenol, prajmaline, propafenone, pyrinoline, quinidine polygalacturonate, quinidine sulfate and viquidil.
6. Antihypertensive Agents

Non-limiting examples of antihypertensive agents include sympatholytic, alpha/beta blockers, alpha blockers, anti-angiotensin II agents, beta blockers, calcium channel blockers, vasodilators and miscellaneous antihypertensives.
a. Alpha Blockers

Non-limiting examples of an alpha blocker, also known as an a-adrenergic blocker or an a-adrenergic antagonist, include amosulalol, arotinolol, dapiprazole, doxazosin, ergoloid mesylates, fenspiride, indoramin, labetalol, nicergoline, prazosin, terazosin, tolazoline, trimazosin and yohimbine. In certain embodiments, an alpha blocker may comprise a quinazoline derivative. Non-limiting examples of quinazoline derivatives include alfuzosin, bunazosin, doxazosin, prazosin, terazosin and trimazosin.
b. Alpha/Beta Blockers

In certain embodiments, an antihypertensive agent is both an alpha and beta adrenergic antagonist. Non-limiting examples of an alpha/beta blocker comprise labetalol (normodyne, trandate).
c. Anti-Angiotension II Agents

Non-limiting examples of anti-angiotension II agents include include angiotensin converting enzyme inhibitors and angiotension II receptor antagonists. Non-limiting examples of angiotension converting enzyme inhibitors (ACE inhibitors) include alacepril, enalapril (vasotec), captopril, cilazapril, delapril, enalaprilat, fosinopril, lisinopril, moveltopril, perindopril, quinapril and ramipril. Non-limiting examples of an angiotensin II receptor blocker, also known as an angiotension II receptor antagonist, an ANG receptor blocker or an ANG-II type-1 receptor blocker (ARBS), include angiocandesartan, eprosartan, irbesartan, losartan and valsartan.
d. Sympatholytics

Non-limiting examples of a syrnpatholytic include a centrally acting sympatholytic or a peripherially acting sympatholytic. Non-limiting examples of a centrally acting sympatholytic, also known as an central nervous system (CNS) sympatholytic, include clonidine (catapres), guanabenz (wytensin) guanfacine (tenex) and methyldopa (aldomet). Non-limiting examples of a peripherally acting sympatholytic include a ganglion blocking agent, an adrenergic neuron blocking agent, a β-adrenergic blocking agent or a alphal-adrenergic blocking agent. Non-limiting examples of a ganglion blocking agent include mecamylamine (inversine) and trimethaphan (arfonad). Non-limiting of an adrenergic neuron blocking agent include guanethidine (ismelin) and reserpine (serpasil). Non-limiting examples of a β-adrenergic blocker include acenitolol (sectral), atenolol (tenormin), betaxolol (kerlone), carteolol (cartrol), labetalol (normodyne, trandate), metoprolol (lopressor), nadanol (corgard), penbutolol (levatol), pindolol (visken), propranolol (inderal) and timolol (blocadren). Non-limiting examples of alphal-adrenergic blocker include prazosin (minipress), doxazocin (cardura) and terazosin (hytrin).
e. Vasodilators

In certain embodiments a cardiovasculator therapeutic agent may comprise a vasodilator (e.g., a cerebral vasodilator, a coronary vasodilator or a peripheral vasodilator). In certain preferred embodiments, a vasodilator comprises a coronary vasodilator. Non-limiting examples of a coronary vasodilator include amotriphene, bendazol, benfurodil hemisuccinate, benziodarone, chloracizine, chromonar, clobenfurol, clonitrate, dilazep, dipyridamole, droprenilamine, efloxate, erythrityl tetranitrane, etafenone, fendiline, floredil, ganglefene, herestrol bis(b-diethylaminoethyl ether), hexobendine, itramin tosylate, khellin, lidoflanine, mannitol hexanitrane, medibazine, nicorglycerin, pentaerythritol tetranitrate, pentrinitrol, perhexiline, pimefylline, trapidil, tricromyl, trimetazidine, trolnitrate phosphate and visnadine.

In certain aspects, a vasodilator may comprise a chronic therapy vasodilator or a hypertensive emergency vasodilator. Non-limiting examples of a chronic therapy vasodilator include hydralazine (apresoline) and minoxidil (loniten). Non-limiting examples of a hypertensive emergency vasodilator include nitroprusside (nipride), diazoxide (hyperstat IV), hydralazine (apresoline), minoxidil (loniten) and verapamil.
f. Miscellaneous Antihypertensives

Non-limiting examples of miscellaneous antihypertensives include ajmaline, g aminobutyric acid, bufeniode, cicletainine, ciclosidomine, a cryptenamine tannate, fenoldopam, flosequinan, ketanserin, mebutamate, mecamylamine, methyldopa, methyl 4-pyridyl ketone thiosemicarbazone, muzolimine, pargyline, pempidine, pinacidil, piperoxan, primaperone, a protoveratrine, raubasine, rescimetol, rilmenidene, saralasin, sodium nitrorusside, ticrynafen, trimethaphan camsylate, tyrosinase and urapidil.

In certain aspects, an antihypertensive may comprise an arylethanolamine derivative, a benzothiadiazine derivative, a N-carboxyalkyl(peptide/lactam) derivative, a dihydropyridine derivative, a guanidine derivative, a hydrazines/phthalazine, an imidazole derivative, a quanternary ammonium compound, a reserpine derivative or a suflonamide derivative.

Arylethanolamine Derivatives. Non-limiting examples of arylethanolamine derivatives include amosulalol, bufuralol, dilevalol, labetalol, pronethalol, sotalol and sulfinalol.

Benzothiadiazine Derivatives. Non-limiting examples of benzothiadiazine derivatives include althizide, bendroflumethiazide, benzthiazide, benzylhydrochlorothiazide, buthiazide, chlorothiazide, chlorthalidone, cyclopenthiazide, cyclothiazide, diazoxide, epithiazide, ethiazide, fenquizone, hydrochlorothizide, hydroflumethizide, methyclothiazide, meticrane, metolazone, paraflutizide, polythizide, tetrachlormethiazide and trichlormethiazide.

N-carboxyalkyl(peptide/lactam) Derivatives. Non-limiting examples of N-carboxyalkyl(peptide/lactam) derivatives include alacepril, captopril, cilazapril, delapril, enalapril, enalaprilat, fosinopril, lisinopril, moveltipril, perindopril, quinapril and ramipril.

Dihydropyridine Derivatives. Non-limiting examples of dihydropyridine derivatives include amlodipine, felodipine, isradipine, nicardipine, nifedipine, nilvadipine, nisoldipine and nitrendipine.

Guanidine Derivatives. Non-limiting examples of guanidine derivatives include bethanidine, debrisoquin, guanabenz, guanacline, guanadrel, guanazodine, guanethidine, guanfacine, guanochlor, guanoxabenz and guanoxan.

Hydrazines/Phthalazines. Non-limiting examples of hydrazines/phthalazines include budralazine, cadralazine, dihydralazine, endralazine, hydracarbazine, hydralazine, pheniprazine, pildralazine and todralazine.

Imidazole Derivatives. Non-limiting examples of imidazole derivatives include clonidine, lofexidine, phentolamine, tiamenidine and tolonidine.

Quanternary Ammonium Compounds. Non-limiting examples of quanternary ammonium compounds include azamethonium bromide, chlorisondamine chloride, hexamethonium, pentacynium bis(methylsulfate), pentamethonium bromide, pentolinium tartrate, phenactropinium chloride and trimethidinium methosulfate.

Reserpine Derivatives. Non-limiting examples of reserpine derivatives include bietaserpine, deserpidine, rescinnamine, reserpine and syrosingopine.

Suflonamide Derivatives. Non-limiting examples of sulfonamide derivatives include ambuside, clopamide, furosemide, indapamide, quinethazone, tripamide and xipamide.
7. Vasopressors

Vasopressors generally are used to increase blood pressure during shock, which may occur during a surgical procedure. Non-limiting examples of a vasopressor, also known as an antihypotensive, include amezinium methyl sulfate, angiotensin amide, dimetofrine, dopamine, etifelmin, etilefrin, gepefrine, metaraminol, midodrine, norepinephrine, pholedrine and synephrine.
8. Treatment Agents For Congestive Heart Failure

Non-limiting examples of agents for the treatment of congestive heart failure include anti-angiotension II agents, afterload-preload reduction treatment, diuretics and inotropic agents.
a. Afterload-Preload Reduction

In certain embodiments, an animal patient that can not tolerate an angiotension antagonist may be treated with a combination therapy. Such therapy may combine adminstration of hydralazine (apresoline) and isosorbide dinitrate (isordil, sorbitrate).
b. Diuretics

Non-limiting examples of a diuretic include a thiazide or benzothiadiazine derivative (e.g., althiazide, bendroflumethazide, benzthiazide, benzylhydrochlorothiazide, buthiazide, chlorothiazide, chlorothiazide, chlorthalidone, cyclopenthiazide, epithiazide, ethiazide, ethiazide, fenquizone, hydrochlorothiazide, hydroflumethiazide, methyclothiazide, meticrane, metolazone, paraflutizide, polythizide, tetrachloromethiazide, trichlormethiazide), an organomercurial (e.g., chlormerodrin, meralluride, mercamphamide, mercaptomerin sodium, mercumallylic acid, mercumatilin dodium, mercurous chloride, mersalyl), a pteridine (e.g., furterene, triamterene), purines (e.g., acefylline, 7-morpholinomethyltheophylline, pamobrom, protheobromine, theobromine), steroids including aldosterone antagonists (e.g., canrenone, oleandrin, spironolactone), a sulfonamide derivative (e.g., acetazolamide, ambuside, azosemide, bumetanide, butazolamide, chloraminophenamide, clofenamide, clopamide, clorexolone, diphenylmethane-4,4′-disulfonamide, disulfamide, ethoxzolamide, furosemide, indapamide, mefruside, methazolamide, piretanide, quinethazone, torasemide, tripamide, xipamide), a uracil (e.g., aminometradine, amisometradine), a potassium sparing antagonist (e.g., amiloride, triamterene)or a miscellaneous diuretic such as aminozine, arbutin, chlorazanil, ethacrynic acid, etozolin, hydracarbazine, isosorbide, mannitol, metochalcone, muzolimine, perhexiline, ticmafen and urea.
c. Other Inotropic Agents

Non-limiting examples of a positive inotropic agent, also known as a cardiotonic, include acefylline, an acetyldigitoxin, 2-amino-4-picoline, amrinone, benfurodil hemisuccinate, bucladesine, cerberosine, camphotamide, convallatoxin, cymarin, denopamine, deslanoside, digitalin, digitalis, digitoxin, digoxin, dobutamine, dopamine, dopexamine, erythrophleine, fenalcomine, gitalin, gitoxin, glycocyamine, heptaminol, hydrastinine, ibopamine, a lanatoside, metamivam, milrinone, nerifolin, oleandrin, ouabain, oxyfedrine, prenalterol, proscillaridine, resibufogenin, scillaren, scillarenin, strphanthin, sulmazole, theobromine and xamoterol.

In particular aspects, an intropic agent is a cardiac glycoside, a beta-adrenergic agonist or a phosphodiesterase inhibitor. Non-limiting examples of a cardiac glycoside includes digoxin (lanoxin) and digitoxin (crystodigin). Non-limiting examples of a β-adrenergic agonist include albuterol, bambuterol, bitolterol, carbuterol, clenbuterol, clorprenaline, denopamine, dioxethedrine, dobutamine (dobutrex), dopamine (intropin), dopexamine, ephedrine, etafedrine, ethylnorepinephrine, fenoterol, formoterol, hexoprenaline, ibopamine, isoetharine, isoproterenol, mabuterol, metaproterenol, methoxyphenamine, oxyfedrine, pirbuterol, procaterol, protokylol, reproterol, rimiterol, ritodrine, soterenol, terbutaline, tretoquinol, tulobuterol and xamoterol. Non-limiting examples of a phosphodiesterase inhibitor include arninone (inocor).
d. Antianginal Agents

Antianginal agents may comprise organonitrates, calcium channel blockers, beta blockers and combinations thereof. Non-limiting examples of organonitrates, also known as nitrovasodilators, include nitroglycerin (nitro-bid, nitrostat), isosorbide dinitrate (isordil, sorbitrate) and amyl nitrate (aspirol, vaporole).
9. Surgical Therapeutic Agents

In certain aspects, the secondary therapeutic agent may comprise a surgery of some type, which includes, for example, preventative, diagnostic or staging, curative and palliative surgery. Surgery, and in particular a curative surgery, may be used in conjunction with other therapies, such as the present invention and one or more other agents.

Such surgical therapeutic agents for vascular and cardiovascular diseases and disorders are well known to those of skill in the art, and may comprise, but are not limited to, performing surgery on an organism, providing a cardiovascular mechanical prostheses, angioplasty, coronary artery reperfusion, catheter ablation, providing an implantable cardioverter defibrillator to the subject, mechanical circulatory support or a combination thereof. Non-limiting examples of a mechanical circulatory support that may be used in the present invention comprise an intra-aortic balloon counterpulsation, left ventricular assist device or combination thereof.

E. Formulations And Routes of Administration For Other Agents

It will be understood that in the discussion of formulations and methods of treatment, references to any compounds are meant to also include the pharmaceutically acceptable salts, as well as pharmaceutical compositions. Where clinical applications are contemplated, pharmaceutical compositions will be prepared in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector or cells, dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present invention, its use in therapeutic compositions is contemplated. Supplementary active ingredients also can be incorporated into the compositions, provided they do not inactivate the vectors or cells of the compositions.

In specific embodiments of the invention the pharmaceutical formulation will be formulated for delivery via rapid release, other embodiments contemplated include but are not limited to timed release, delayed release, and sustained release. Formulations can be an oral suspension in either the solid or liquid form. In further embodiments, it is contemplated that the formulation can be prepared for delivery via parenteral delivery, or used as a suppository, or be formulated for subcutaneous, intravenous, intramuscular, intraperitoneal, sublingual, transdermal, or nasopharyngeal delivery.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in the U.S. Pat. Nos. 4,256,108; 4,166,452; and 4,265,874 to form osmotic therapeutic tablets for control release (hereinafter incorporated by reference).

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain an active material in admixture with excipients suitable for the manufacture of aqueous suspensions. Such excipients are suspending agents, for example sodium carboxymethylcellulose, methylcellulose, hydroxypropylmethycellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavoring and coloring agents. Pharmaceutical compositions may be in the form of a sterile injectable aqueous or oleagenous suspension. Suspensions may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds may also be administered in the form of suppositories for rectal administration of the drug. These compositions can be prepared by mixing a therapeutic agent with a suitable non-irritating excipient which is solid at ordinary temperatures, but liquid at the rectal temperature and will therefore melt in the rectum to release the drug. Such materials are cocoa butter and polyethylene glycols.

For topical use, creams, ointments, jellies, gels, epidermal solutions or suspensions, etc., containing a therapeutic compound are employed. For purposes of this application, topical application shall include mouthwashes and gargles.

Formulations may also be administered as nanoparticles, liposomes, granules, inhalants, nasal solutions, or intravenous admixtures

The previously mentioned formulations are all contemplated for treating patients suffering from heart failure or hypertrophy. The amount of active ingredient in any formulation may vary to produce a dosage form that will depend on the particular treatment and mode of administration. It is further understood that specific dosing for a patient will depend upon a variety of factors including age, body weight, general health, sex, diet, time of administration, route of administration, rate of excretion, drug combination and the severity of the particular disease undergoing therapy.

V. Definitions

As used herein, the term “heart failure” is broadly used to mean any condition that reduces the ability of the heart to pump blood. As a result, congestion and edema develop in the tissues. Most frequently, heart failure is caused by decreased contractility of the myocardium, resulting from reduced coronary blood flow; however, many other factors may result in heart failure, including damage to the heart valves, vitamin deficiency, and primary cardiac muscle disease. Though the precise physiological mechanisms of heart failure are not entirely understood, heart failure is generally believed to involve disorders in several cardiac autonomic properties, including sympathetic, parasympathetic, and baroreceptor responses. The phrase “manifestations of heart failure” is used broadly to encompass all of the sequelae associated with heart failure, such as shortness of breath, pitting edema, an enlarged tender liver, engorged neck veins, pulmonary rales and the like including laboratory findings associated with heart failure.

The term “treatment” or grammatical equivalents encompasses the improvement and/or reversal of the symptoms of heart failure (i.e., the ability of the heart to pump blood). “Improvement in the physiologic function” of the heart may be assessed using any of the measurements described herein (e.g., measurement of ejection fraction, fractional shortening, left ventricular internal dimension, heart rate, etc.), as well as any effect upon the animal's survival. In use of animal models, the response of treated transgenic animals and untreated transgenic animals is compared using any of the assays described herein (in addition, treated and untreated non-transgenic animals may be included as controls). A compound which causes an improvement in any parameter associated with heart failure used in the screening methods of the instant invention may thereby be identified as a therapeutic compound.

The terms “compound” and “chemical agent” refer to any chemical entity, pharmaceutical, drug, and the like that can be used to treat or prevent a disease, illness, sickness, or disorder of bodily finction. Compounds and chemical agents comprise both known and potential therapeutic compounds. A compound or chemical agent can be determined to be therapeutic by screening using the screening methods of the present invention. A “known therapeutic compound” refers to a therapeutic compound that has been shown (e.g., through animal trials or prior experience with administration to humans) to be effective in such treatment. In other words, a known therapeutic compound is not limited to a compound efficacious in the treatment of heart failure.

As used herein, the term “cardiac hypertrophy” refers to the process in which adult cardiac myocytes respond to stress through hypertrophic growth. Such growth is characterized by cell size increases without cell division, assembling of additional sarcomeres within the cell to maximize force generation, and an activation of a fetal cardiac gene program. Cardiac hypertrophy is often associated with increased risk of morbidity and mortality, and thus studies aimed at understanding the molecular mechanisms of cardiac hypertrophy could have a significant impact on human health.

As used herein, the terms “antagonist” and “inhibitor” refer to molecules, compounds, or nucleic acids which inhibit the action of a cellular factor that may be involved in heart failure or cardiac hypertrophy. Antagonists may or may not be homologous to these natural compounds in respect to conformation, charge or other characteristics. Thus, antagonists may be recognized by the same or different receptors that are recognized by an agonist. Antagonists may have allosteric effects which prevent the action of an agonist. Alternatively, antagonists may prevent the function of the agonist. In contrast to the agonists, antagonistic compounds do not result in pathologic and/or biochemical changes within the cell such that the cell reacts to the presence of the antagonist in the same manner as if the cellular factor was present. Antagonists and inhibitors may include proteins, nucleic acids, carbohydrates, or any other molecules which bind or interact with a receptor, molecule, and/or pathway of interest.

As used herein, the term “modulate” refers to a change or an alteration in a biological activity. Modulation may be an increase or a decrease in protein activity, a change in kinase activity, a change in binding characteristics, or any other change in the biological, functional, or immunological properties associated with the activity of a protein or other structure of interest. The term “modulator” refers to any molecule or compound which is capable of changing or altering biological activity as described above.

VI. Examples

Pharmacokinetic (PK) values. At various times after dosing of enoximone specimens were collected and prepared for analysis. Time points included pre-dosing or 0 hours, 0.5 hours, 2 hours, 4 hours, 6 hours and 8 hours (also a pre-dosing time point). Blood samples were colleceted in Royal Blue Na2 EDTA 7.0 ml tubes, placed in an ice bath and centrifuged, then placed back in an ice bath and plasma was transferred via plastic pipette to green tubes (1 mL) and stored at −20C until analyzed.

Samples were then prepared by an automated solid-phase extraction procedure and analyzed by liquid chromatography/tandem mass spectrometry. The API 4000 was operated in the multiple reaction monitoring (MRM) mode under optimized conditions for detection of enoximone and enoximone sulfoxide. Each run included calibration standards in duplicate with at least seven different concentrations. Dilution QB samples were analyzed in replicated of six. The lower limit of quantitation (LLOQ) of the assay in human plasma was determined at 1.00 ng/mL for both enoximone and enoximone sulfoxide. Derived PK values shown in Table 1, 2 and 3 are derived from two different patient populations.

TABLE 1
PK Values Experiment (23 patients analyzed, mean data)
PK Values Experiment 1

| | 0 hr | 2 hrs | 4 hrs | 8 hrs |
|---|---|---|---|---|
| [E] (ng/ml) | | | | |
| Min | 0 | 20 | 15 | 0 |
| Max | 150 | 400 | 200 | 150 |
| [ES] (ng/ml) | | | | |
| Mm | 40 | 80 | 80 | 40 |
| Max | 800 | 1500 | 1500 | 800 |

TABLE 2
PK Values Experiment 2 (5 patients analyzed, raw data)
PK Values Experiment 2

| | 0 hr | 2 hrs | 4 hrs | 8 hrs |
|---|---|---|---|---|
| [E] (ng/ml) | | | | |
| Min | 7.95 | 63.6 | 44 | 20.7 |
| Max | 149 | 383 | 268 | 166 |
| [ES] (ng/ml) | | | | |
| Min | 66.7 | 344 | 219 | 129 |
| Max | 794 | 1540 | 1310 | 1070 |

TABLE 3
PK Values Experiment (5 patients analyzed, mean data)

| | Dose | | | |
|---|---|---|---|---|
| Patient | (mg) | Sample Time | [E] (ng/ml) | [E-S] (ng/ml) |
| 1 | 50 | PREDOSE ALQOT 1 | 69.7 | 665 |
| | | 0.5 HR ALIQUOT 1 | 118 | 706 |
| | | 1 HR ALIQUOT 1 | 424 | 1440 |
| | | 2 HR ALIQUOT 1 | 383 | 1540 |
| | | 4 HR ALIQUOT 1 | 209 | 1310 |
| | | 6 HR ALIQUOT 1 | 112 | 961 |
| 2 | 50 | PREDOSE ALQOT 1 | 66.7 | 607 |
| | | 0.5 HR ALIQUOT 1 | 52.8 | 608 |
| | | 1 HR ALIQUOT 1 | 49.8 | 540 |
| | | 2 HR ALIQUOT 1 | 127 | 600 |
| | | 4 HR ALIQUOT 1 | 237 | 1290 |
| | | 6 HR ALIQUOT 1 | 122 | 1070 |
| 3 | 50 | PREDOSE ALQOT 1 | 34.4 | 302 |
| | | 0.5 HR ALIQUOT 1 | 92.4 | 472 |
| | | 1 HR ALIQUOT 1 | 118 | 867 |
| | | 2 HR ALIQUOT 1 | 110 | 758 |
| | | 4 HR ALIQUOT 1 | 78.2 | 616 |
| | | 6 HR ALIQUOT 1 | 71.7 | 445 |
| 4 | 25 | PREDOSE ALQOT 1 | 18.6 | 98.3 |
| | | 0.5 HR ALIQUOT 1 | 23.7 | 113 |
| | | HR ALIQUOT 1 | 65.8 | 235 |
| | | 2 HR ALIQUOT 1 | 63.6 | 344 |
| | | 4 HR ALIQUOT 1 | N/A | N/A |
| | | 6 HR ALIQUOT 1 | N/A | N/A |
| 5 | 50 | PREDOSE ALQOT 1 | 149 | 794 |
| | | 0.5 HR ALIQUOT 1 | 137 | 777 |
| | | 1 HR ALIQUOT 1 | 355 | 958 |
| | | 2 HR ALIQUOT 1 | 369 | 1240 |
| | | 4 HR ALIQUOT 1 | 268 | 1290 |
| | | 6 HR ALIQUOT 1 | 166 | 960 |

VII. References

The following references, to the extent that they provide exemplary procedural or other details supplementary to those set forth herein, are specifically incorporated herein by reference.
U.S. Pat. No. 4,166,452
U.S. Pat. No. 4,256,108
U.S. Pat. No. 4,265,874
U.S. Pat. No. 4,505,635
U.S. Pat. No. 5,604,251
U.S. Pat. No. 5,998,458
U.S. Pat. No. 6,254,885
U.S. Pat. No. 6,555,135
U.S. Pat. No. 6,596,308
U.S. Pat. No. 6,623,760
U.S. Pat. No. 6,645,466
EP Patent 326103
Ahmed, Am. Geriatr. Soc., 51 (1):123-126, 2003.
Bristow, Z. Kardiol., 83 (2):15-19, 1994.
Bristow, Cardiology, 92:3-6, 1999.
Conte et al., J. Heart Lung Transplant., 17:679-685, 1998.
De Feo et al., Ital. Heart J.,4:511-513, 2003.
DiBianco, Am. J. Med., 115:480-488, 2003.
Dumcius et al., Medicina, 39:815-822, 2003.
Durand et al., Ann. Med., 27:311-317, 1995.
Eichhom and Bristow, Circulation, 94:2285-2296, 1996.
Goodman and Gilman's The Pharmacological Basis Of Therapeutics, Hardman et al. (Eds.), 10^{th} ed., 32:853-860; 35:891-893, 2001.
Handbook of Pharmaceutical Excipients, 3d Ed., Kibbe (Ed.), 2000.
Handbook of Industrial Surfactants, 3d Ed. Vol. 1 & 2, Ash (Ed.), 2000.
www.myogen.com/pipeline/enoximone.php, last visited Dec. 9, 2004.
Levy et al., N. Engl. J. Med., 322:1561-1566, 1990.
Manoria and Manoria, J. Indian. Med. Assoc., 101 (5):311-312, 2003.
Merck Index, Thirteenth Edition
Om & Hess, Clin. Cardiol., 16 (1):5-14, 1993.
PCT Appl. WO 98/33791
Physicians Desk Reference.
Young et al., Handbook of Applied Therapeutics, 7.1-7.12 and 9.1-9.10, 1989.

1. A pharmaceutical formulation comprising enoximone, about 40-80% non-ionic surfactant by weight, and wherein the primary particle size of said enoximone is less than 10 μm on average.

2-101. (canceled)

102. The pharmaceutical formulation of claim 1, wherein the non-ionic surfactant is Polysorbate-80 present at about 66% and wherein the primary particle size of said enoximone is less than 10 μm on average, and wherein said formulation yields at chronic dosing a blood level of enoximone of about 1-160 ng/ml immediately after dosing, about 20-500 ng/ml at about 2 hours post dosing, about 15-300 ng/ml at about 4 hours post dosing, and about 1-200 ng/ml at 8 hours post dosing.

103. The pharmaceutical formulation of claim 1, wherein the non-ionic surfactant is Polysorbate-80 present at about 66% and enoximone, wherein the primary particle size of said enoximone is less than 10 μm on average, and wherein said formulation yields at chronic dosing a blood level of enoximone sulfoxide of about 40-900 ng/ml immediately after dosing, 80-1700 ng/ml at about 2 hours post dosing, 80-1700 ng/ml at about 4 hours post dosing, and 40-1200 ng/ml at about 8 hours post dosing.

104. The pharmaceutical formulation of claim 1, wherein said enoximone consists of either 25 milligrams or 50 milligrams.

105. The pharmaceutical formulation of claim 1, wherein said particle size is, on average, less than about 9 microns, less than about 8 microns, less than about 7 microns, less than about 6 microns, less than about 5 microns, less than about 4 microns, less than about 3 microns, less than about 2 microns, or less than about 1 micron.

106. A pharmaceutical formulation of enoximone comprising about 66% Polysorbate-80 and enoximone, wherein the primary particle size of said enoximone is less than 10 μm on average, and wherein said formulation yields on chronic dosing, a blood level of enoximone immediately after dosing of 1-150 ng/ml, 5-160 ng/ml, 5-150 ng/ml, 7-149 ng/ml, or 7-150 ng/ml; a blood level of enoximone at about 2 hours post dosing of 20-400 ng/ml, 30-500 ng/ml, 30-400 ng/ml, 60-385 ng/ml, or 50-390 ng/ml; a blood level of enoximone at about 4 hours post dosing of 15-200 ng/ml, 20-300 ng/ml, 20-200 ng/ml, or 40-270 ng/ml; and a blood level of enoximone at about 8 hours post dosing of 1-150 ng/ml, 5-200 ng/ml, or 5-150 ng/ml.

107. A pharmaceutical formulation of enoximone comprising about 66% Polysorbate-80 and enoximone, wherein the primary particle size of said enoximone is less than 10 μm on average, and wherein said formulation yields on chronic dosing a blood level of enoximone sulfoxide immediately post dosing of 40-800 ng/ml, 50-900 ng/ml, 50-800 ng/ml, or 60-800 ng/ml; a blood level of enoximone sulfoxide at about 2 hours post dosing of 80-1500 ng/ml, 200-1700 ng/ml, 200-1500 ng/ml, 300-1500 ng/ml, 250-1600 ng/ml, or 300-1550 ng/ml; a blood level of enoximone sulfoxide at about 4 hours post dosing of 80-1500 ng/ml, 150-1500 ng/ml, or 200-1350 ng/ml; and a blood level of enoximone sulfoxide at about 8 hours post dosing of 40-800 ng/ml, 50-900 ng/ml, 90-1200 ng/ml, or 50-800 ng/ml.

108. The pharmaceutical formulation of claim 106, wherein said enoximone consists of either 25 milligrams or 50 milligrams.

109. The pharmaceutical formulation of claim 107, wherein said enoximone consists of either 25 milligrams or 50 milligrams.

110. The pharmaceutical formulation of claim 106, wherein said formulation yields on chronic dosing, a blood level of enoximone immediately after dosing of 1-150 ng/ml; a blood level of enoximone at about 2 hours post dosing of 20-400 ng/ml; a blood level of enoximone at about 4 hours post dosing of 15-200 ng/ml; and a blood level of enoximone at about 8 hours post dosing of 1-150 ng/ml.

111. The pharmaceutical formulation of claim 106, wherein said formulation yields on chronic dosing, a blood level of enoximone immediately after dosing of 5-160 ng/ml; a blood level of enoximone at about 2 hours post dosing of 30-500 ng/ml; a blood level of enoximone at about 4 hours post dosing of 20-300 ng/ml; and a blood level of enoximone at about 8 hours post dosing of 5-200 ng/ml.

112. The pharmaceutical formulation of claim 106, wherein said formulation yields on chronic dosing, a blood level of enoximone immediately after dosing of 5-150 ng/ml; a blood level of enoximone at about 2 hours post dosing of 30-400 ng/ml; a blood level of enoximone at about 4 hours post dosing of 20-200 ng/ml; and a blood level of enoximone at about 8 hours post dosing of 5-150 ng/ml.

113. The pharmaceutical formulation of claim 106, wherein said formulation yields on chronic dosing, a blood level of enoximone immediately after dosing of 7-149 ng/ml; a blood level of enoximone at about 2 hours post dosing of 60-385 ng/ml; a blood level of enoximone at about 4 hours post dosing of 40-270 ng/ml; and a blood level of enoximone at about 8 hours post dosing of 5-150 ng/ml.

114. The pharmaceutical formulation of claim 106, wherein said formulation yields on chronic dosing, a blood level of enoximone immediately after dosing of 7-150 ng/ml; a blood level of enoximone at about 2 hours post dosing of 50-390 ng/ml; a blood level of enoximone at about 4 hours post dosing of 40-270 ng/ml; and a blood level of enoximone at about 8 hours post dosing of 1-150 ng/ml.

115. The pharmaceutical formulation of claim 107, wherein said formulation yields on chronic dosing a blood level of enoximone sulfoxide immediately post dosing of 40-800; a blood level of enoximone sulfoxide at about 2 hours post dosing of 80-1500 ng/ml; a blood level of enoximone sulfoxide at about 4 hours post dosing of 80-1500 ng/ml; and a blood level of enoximone sulfoxide at about 8 hours post dosing of 40-800 ng/ml.

116. The pharmaceutical formulation of claim 107, wherein said formulation yields on chronic dosing a blood level of enoximone sulfoxide immediately post dosing of 50-900 ng/ml; a blood level of enoximone sulfoxide at about 2 hours post dosing of 200-1700 ng/ml; a blood level of enoximone sulfoxide at about 4 hours post dosing of 150-1500 ng/ml; and a blood level of enoximone sulfoxide at about 8 hours post dosing of 50-900 ng/ml.

117. The pharmaceutical formulation of claim 107, wherein said formulation yields on chronic dosing a blood level of enoximone sulfoxide immediately post dosing of 50-800 ng/ml; a blood level of enoximone sulfoxide at about 2 hours post dosing of 200-1500 ng/ml; a blood level of enoximone sulfoxide at about 4 hours post dosing of 200-1350 ng/ml; and a blood level of enoximone sulfoxide at about 8 hours post dosing of 90-1200 ng/ml.

118. The pharmaceutical formulation of claim 107, wherein said formulation yields on chronic dosing a blood level of enoximone sulfoxide immediately post dosing of 60-800 ng/ml; a blood level of enoximone sulfoxide at about 2 hours post dosing 300-1500 ng/ml; a blood level of enoximone sulfoxide at about 4 hours post dosing of 200-1350 ng/ml; and a blood level of enoximone sulfoxide at about 8 hours post dosing of 50-800 ng/ml.

119. The pharmaceutical formulation of claim 106, wherein said particle size is, on average, less than about 9 microns, less than about 8 microns, less than about 7 microns, less than about 6 microns, less than about 5 microns, less than about 4 microns, less than about 3 microns, less than about 2 microns, or less than about 1 micron.

120. The pharmaceutical formulation of claim 107, wherein said particle size is, on average, less than about 9 microns, less than about 8 microns, less than about 7 microns, less than about 6 microns, less than about 5 microns, less than about 4 microns, less than about 3 microns, less than about 2 microns, or less than about 1 micron.
